(19) United States
(12) Patent Application Publication
(10) Pub. No.: US 2022/0379064 A1
(43) Pub. Date: Dec. 1, 2022

(54) Assembly for conducting respiratory air in a patient ventilation system

(71) Applicant: RAUMEDIC AG, Münchberg (DE)

(72) Inventor: Björn GLÄSEL, Kirchenlamitz (DE)

(73) Assignee: RAUMEDIC AG, Münchberg (DE)

(21) Appl. No.: 17/771,409

(22) PCT Filed: Oct. 8, 2020
(86) PCT No.: PCT/EP2020/078216

§ 371 Date: Apr. 22, 2022

(30) Foreign Application Priority Data
Oct. 25, 2019 (DE) 10 2019 216 506.0

(51) Int. Cl. A61M 16/08; A61M 16/10

(52) U.S. Cl. CPC A61M 16/0816; A61M 16/1095; A61M 16/0875

(57) ABSTRACT

An assembly for conducting respiratory air in a patient ventilation system has a plastic respiratory air tube component for conducting respiratory air. At least one heating wire is used to heat the respiratory air. A plastic connector conducts the respiratory air, is integrally connected to an end portion of the tube component and is used for connection to a further, external respiratory air-conducting component. The connector has a connection piece in which an end portion of the heating wire is electrically contacted to an external supply line. The end portion of the heating wire is routed through a lateral wall of the connector to the connection piece. In the connection piece, a recess for accommodating a protective plastic housing for an electrical contacting portion is provided between the heating wire end section and the end portion of the supply line.

### CROSS-REFERENCES TO RELATED APPLICATIONS

This application claims the priority of German Patent Application, Serial No. DE 10 2019 216 506.0, filed Oct. 25, 2019, the content of which is incorporated herein by reference in its entirety as if fully set forth herein.

### FIELD OF THE INVENTION

The invention relates to an assembly for conducting respiratory air in a patient ventilation system.

### BACKGROUND OF THE INVENTION

An assembly for conducting respiratory air is generally known from U.S. Pat. No. 10,213,571 B2.

### SUMMARY OF THE INVENTION

It is an object of the present invention to further design an assembly for conducting respiratory air of the type mentioned above in such a manner that an integral connection of a connector of the assembly to an end portion of a tube component of the assembly can be securely established.

This object is achieved according to the invention by an assembly for conducting respiratory air in a patient ventilation system
having a respiratory air tube component with a tube wall made of plastic, for conducting respiratory air,
having at least one heating wire to heat the respiratory air,
having a connector, which conducts the respiratory air, is made of plastic and is integrally connected to an end portion of the tube component, for connecting the tube component to a further, external respiratory air-conducting component,
wherein the connector has a connection piece integrally connected thereto, in which an end portion of the heating wire is electrically contacted to a supply line,
wherein the end portion of the heating wire is routed through a lateral wall of the connector to the connection piece,
having a protective plastic housing for an electrical contacting portion between the heating wire end portion and an end portion of the supply line electrically contacted thereto,
wherein a recess for accommodating the protective housing is provided in the connection piece.

According to the invention, it has been recognized that a protective housing for an electrical contacting portion between end portions of a heating wire on the one hand and a supply line on the other hand can be used to protect the contacting portion when connecting the tube component to the connector, in particular when overmolding the tube component with the connector. Electrical contacting between the heating wire end portion and the supply line end portion electrically contacted thereto may be performed by soldering or crimping. The protective housing, insofar as the integral connection is established by overmolding, is also overmolded. The protective housing can be designed in such a way that plastic overmolding material cannot penetrate into the protective housing. The supply line as a component of the assembly can in turn be connected to an external electrical supply device, in particular via a plug connection.

An embodiment in which the heating wire is configured in the form of a plastic-coated heating coil around an inner lumen of the tube component is readily realizable in terms of production technology. The heating coil can be an outer wall profiling of the tube component, which is particularly suitable for realizing an integral connection to the connector, in particular by overmolding.

A design of the connection piece configured such that the connection piece extends radially from a longitudinal axis of the connector has proven to be effective. Alternatively, it is possible to design the connection piece set at an angle with respect to a connector longitudinal axis. This is advantageous in situations where a supply line for the heating wire is to be laid along the tube component under a preferred direction.

A design of the protective housing in which the protective housing is configured such that a plurality of heating wire end portions are electrically contacted therein to associated supply line end portions takes into account the design of typical tube component heating units.

A separating web of the protective housing for insulation between the heating wire end portions and/or the supply line end portions ensures an insulation between filaments of the heating wire supply to be insulated from each other.

A cover which is connected to a base body of the protective housing improves a handling and a protective effect of the protective housing. The cover may be integrally molded to the base body of the protective housing.

A solid-body hinge between the cover and the base body results in a protective housing that can be manufactured inexpensively. A closed position between the cover and the base body can be configured as a latched closed position.

An overmolding body which is arranged such as to support a tube wall of the end portion from the inside when the connector is molded to the end portion of the tube component prevents undesirable damage to the tube wall of the end portion of the tube component when the integral connection between the tube component and the connector is established, in particular during overmolding. The overmolding body may be in the form of a grommet which is inserted into the end portion of the tube component which is adjacent to or overlapping with the connector. The overmolding body may overlap with the connector in portions.

A radially projecting collar of the overmolding body enables secure relative axial positioning between the overmolding body on the one hand and the end portion of the tube component to be integrally connected to the connector on the other hand. The collar can be inserted into a complementary peripheral recess in an inner wall of the connector.

Examples of embodiments of the invention are explained in more detail below with reference to the drawing.

### BRIEF DESCRIPTION OF THE DRAWING

FIG. 1 schematically shows main components of a patient ventilation system including a main unit, a humidifier, a respiratory mask, and a plurality of heatable tube components connecting them and conducting the respiratory air, including a plurality of connectors for fluid-conducting connection of these components;

FIG. 2 shows a perspective view of main components of an assembly for conducting respiratory air of the ventilation system of FIG. 1, namely an end portion of one of the tube components, a connector, and a supply line portion with a connector plug component;

FIG. 3 more schematically shows an axial section through the assembly according to FIG. 2, wherein a supply line end portion, which is electrically connected to a heating wire of the respiratory air tube component when the assembly is manufactured, is shown in the region of an electrical contacting portion still spaced apart from the facing heating wire end portion;

FIG. 4 shows the electrical contacting portion between the heating wire end portion and the supply line end portion electrically contacted therewith, arranged in a plastic protective housing;

FIG. 5 shows a top view onto the protective housing with the cover hinged open relative to a base body;

FIG. 6 shows a view according to direction VI in FIG. 5;

FIG. 7 in a view similar to FIG. 3, shows another embodiment of an assembly having an end portion of a respiratory air tube component and a connector, wherein a connection piece integrally connected to the connector is radially attached to a base body of the connector; and

FIG. 8 shows a side view of an assembly alternative to the assembly according to FIGS. 3 and 7, still without a supply line, wherein a connector including a connection piece is shown cut open.

### DESCRIPTION OF THE PREFERRED EMBODIMENTS

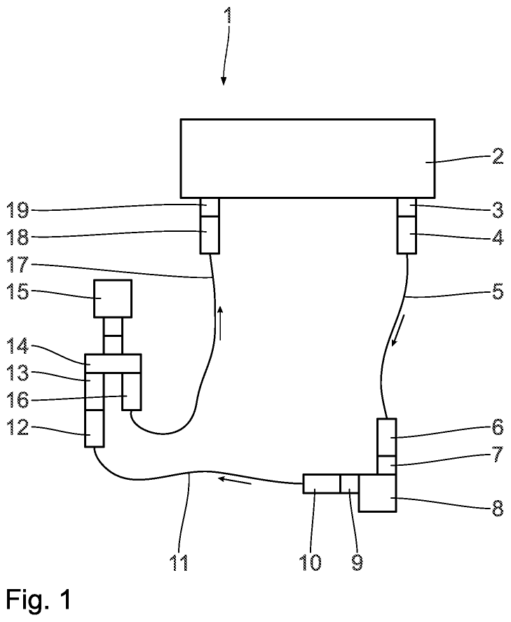

A patient ventilation system 1, the main components of which are shown in FIG. 1, serves for the ventilation of a patient in a clinical, other inpatient or even home care setting.

The ventilation system has a main unit 2 for controlling/regulating, in particular, a respiratory air supply, air humidification and temperature control of the respiratory air. In principle, the main unit 2 may also serve to determine a respiratory air composition. The main unit 2 serves as a respiratory air source.

The main unit 2 is in fluid connection with a respiratory air supply tube component 5 via a junction port 3 and a junction connector 4. The latter is shown very schematically in FIG. 1, as are the other tube components. The tube components of the ventilation system 1 can be heated in a controlled/regulated manner via the main unit 2, as will be described below.

Via a further junction connector 6, the respiratory air supply tube component is in fluid connection with a junction port 7 of a respiratory air humidification device 8. The humidification device 8 is in signal communication with the main unit 2 in a manner not shown.

The humidification device 8 is in fluid connection with a respiratory air connection tube component 11 via a further junction port 9 and a further junction connector 10.

The respiratory air connection tube component 11 is in fluid connection with a patient respiratory mask 15 via a further junction connector 12, a monitoring connector 13 and a three-way connector 14. The monitoring connector 13 thus, on the one hand, serves to connect to the respiratory air connection tube component 11, i.e. to a heatable respiratory air tube portion for conducting ventilation air from the main unit 2 to the patient, and, on the other hand, to connect this tube component 11 to the patient respiratory mask 15 as a patient air interface.

The respiratory mask 15, via the three-way connector 14 and a further junction connector 16, is in fluid connection with a respiratory air discharge tube component 17. The latter is in fluid connection with the main unit 2 via a further junction connector 18 and a further junction port 19.

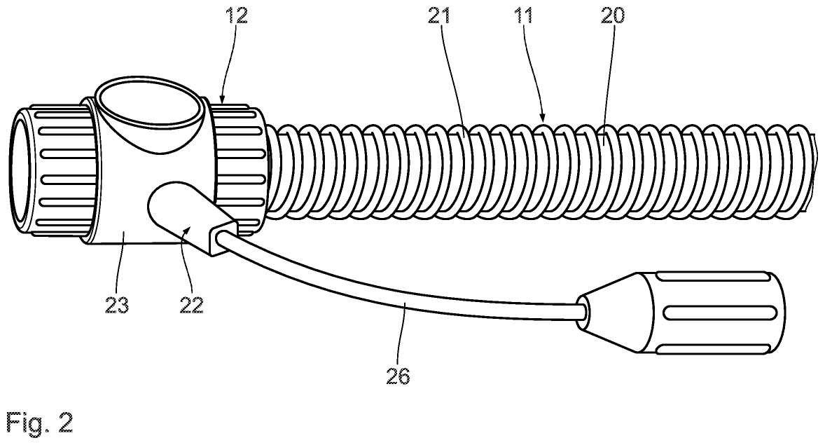

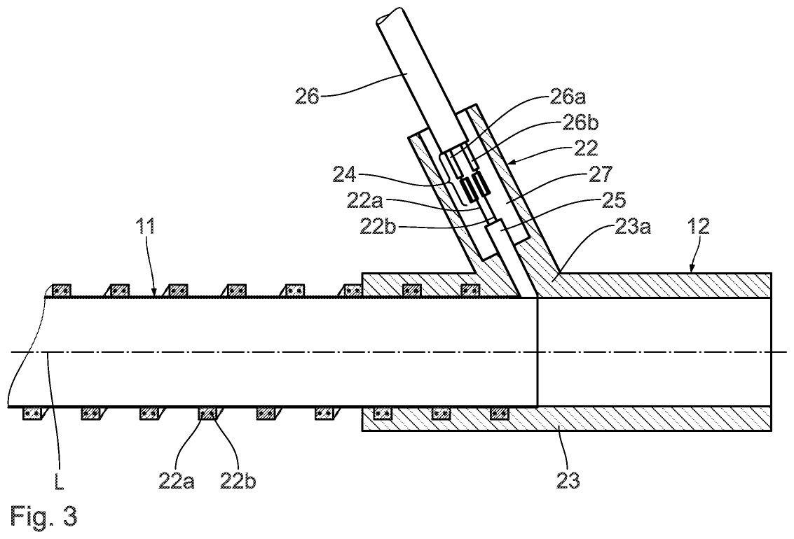

FIG. 2 shows a portion of the respiratory air connection tube component 11 together with the junction connector 12. The respiratory air tube component has a tube wall 20 made of plastic, transparent in FIG. 2, for conducting the respiratory air. Two heating wires 22a, 22b (cf. FIG. 3 ff) are routed in a heating coil 21, which is wound around the tube wall 20 to form the integral respiratory air tube component 11, which serve to heat the respiratory air conducted through the respiratory air tube component 11. The two heating wires 22a, 22b may represent different phases of the electrical supply. The heating wire 22a/b is configured in the form of a plastic-coated heating coil around an inner lumen of the tube component 11.

The connector 12 is integrally connected to an end portion of the tube component 11 by overmolding. The connector 12 serves to connect the tube component 11 to a further, external respiratory air-conducting component, which is not shown in the drawing.

The connector 12 has a connection piece 22 which is integrally connected to a base body 23 of the connector 12. The connection piece 22 extends obliquely away from the base body 23 of the connector 12, with respect to a longitudinal axis L of the connector 12, which coincides with a longitudinal axis of the facing end portion of the tube component 11.

FIG. 3 shows details of a structure of the assembly with the tube component 11 and the connector 12 including the connection piece 22 in a sectional view showing further details on the one hand, which is more schematic than the one shown in FIG. 2 on the other hand.

The connection piece 22 is provided for accommodating an electrical contacting portion 24 between an end portion 25 of the heating wires 22a/b and an end portion of a supply line 26 electrically contacted to this heating wire end portion 25. For this purpose, the connection piece 22 has a recess 27.

The heating wire end portion 25 is routed through a lateral wall 23a of the connector 12 to the connection piece 22.

The assembly for conducting respiratory air with the tube component 11 and the connector 12 also includes the supply line 26.

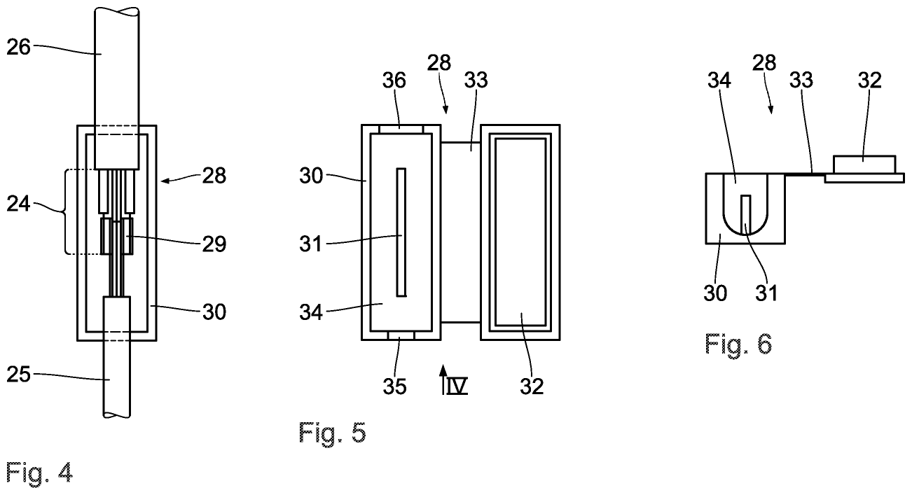

The recess 27 in the connection piece 22 serves to accommodate a protective plastic housing 28 for the electrical contacting portion 24 (cf. FIGS. 4 to 6). The protective housing 28 is designed such that end portions of the heating wires 22a/b are electrically contacted therein to associated end portions 26a/b of the supply line 26 via a line connection 29. This electrical contacting can be carried out by soldering and/or crimping.

A base body 30 of the protective housing 28 has a separating web 31 for insulation between the heating wire/supply line end portions 22a/26a contacted to each other on the one hand and the heating wire/supply line end portions 22b/26b contacted to each other on the other hand. The separating web 31 prevents the two heating wires 22a and 22b on the one hand and the two supply lines 26a, 26b on the other hand from undesirably coming into contact with each other in regions where they are stripped to create the line connection 29.

The protective housing 28 has a cover 32 that is connected to the base body of the protective housing 28 via a solid-body hinge 33, namely is integrally formed therewith. A latched closed position may be present between the cover 32 and the base body 30. The base body 30, on the one hand, and the cover 32, on the other hand, delimit an accommodation space 34 for accommodating the electrical contacting portion 24. For routing out insulated lead portions of the heating wire end portion 25, on the one hand, and the end portion of the supply line 26, on the other hand, respective passage openings 35, 36 are formed in end walls of the base body 30.

In the production of the assembly according to FIG. 2 or 3, the connector 12 is first pushed onto the end portion of the tube component 11, wherein a screw thread complementary to the heating coil may be formed in an inner wall of the base body 23, so that after the insertion of the end portion of the tube component 11, which in this case takes place by screwing in, there is already a form-fit mechanical connection between these two components. During this insertion or following same, the heating wire end portion 25 is routed in the connection piece 22 as far as the recess 27. Electrical contact is then established between the heating wires 22a/b of the heating wire end portion 25 and the associated supply lines 26a/b via the line connection 29. After that, the protective housing 28 is inserted into the recess 27 with the cover open until the contacting portion 24 comes to rest completely in the accommodation space 34 of the base body 30. The protective housing is then closed by closing the cover 32. In this configuration, the assembly including the tube component 11, the connector 12, the supply line 26 and the protective housing 28 can be overmolded with plastic material so that an integral connection of these components to one another is provided.

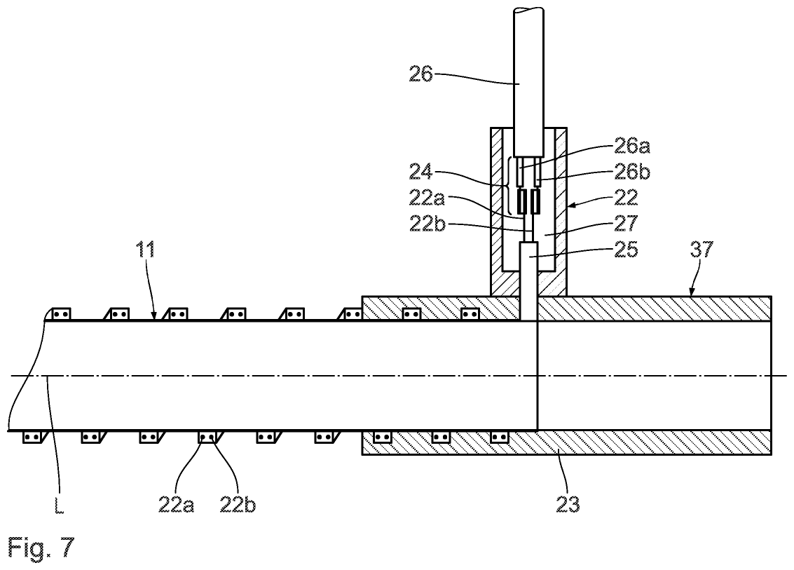

FIG. 7 shows an assembly having an end portion of a tube component 11 and a connector 37, which can be used as an alternative to the connector 12 shown in FIGS. 1 to 6. Components and functions corresponding to those already explained above with reference to FIGS. 1 to 6 bear the same reference numerals and will not be discussed in detail again. In the connector 37, the connection piece 22 extends radially from the longitudinal axis L of the connector 37.

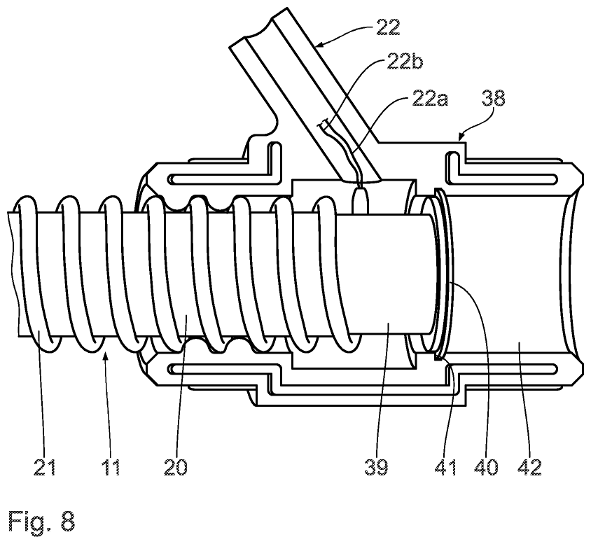

With reference to FIG. 8, a further embodiment of an overmolding connection between an end portion of a tube component 11 and a connector 38, which can be used instead of the connectors 12 and 37, is described below. Components and functions corresponding to those already explained above with reference to FIGS. 1 to 7 bear the same reference numerals and will not be discussed in detail again.

The assembly with the connector 38 and the end portion of the tube component 11 facing same includes an overmolding body 39, which is arranged in such a way that it supports the tube wall 20 of the end portion from the inside when the connector 38 is molded onto the end portion of the tube component 11. For this purpose, the overmolding body 39 is designed as a grommet and has a radially projecting collar 40, which serves to axially fix the overmolding body 39 in the connector 38 during overmolding. For the axial fixation of the overmolding body 39, the collar is located in a complementary circumferential recess 41 in an inner wall 42 of the connector 38 when the overmolding body 39 is inserted.

In the inserted position shown in FIG. 8, the overmolding body 39 overlaps with the connector 38 in the axial direction.

When overmolding the connector 38 and the associated end portion of the tube component 11, the overmolding body 39 ensures that the tube wall 20, which is softer due to the increased overmolding temperature, is securely supported from the inside and is not damaged by the overmolding process.

1. An assembly for conducting respiratory air in a patient ventilation system
having a respiratory air tube component (11) with a tube wall (20) made of plastic, for conducting respiratory air,
having at least one heating wire (22a, 22b) to heat the respiratory air,
having a connector (12; 37; 38), which conducts the respiratory air, is made of plastic and is integrally connected to an end portion of the tube component (11), for connecting the tube component (11) to a further, external respiratory air-conducting component,
wherein the connector (12; 37; 38) has a connection piece (22) integrally connected thereto, in which an end portion (25) of the heating wire (22a, 22b) is electrically contacted to a supply line (26),
wherein the end portion (25) of the heating wire (22a, 22b) is routed through a lateral wall (23a) of the connector (12; 37; 38) to the connection piece (22),
having a protective plastic housing (28) for an electrical contacting portion (24) between the heating wire end portion (25) and an end portion of the supply line (26) electrically contacted thereto,
wherein a recess (27) for accommodating the protective housing (28) is provided in the connection piece (22).

2. The assembly according to claim 1, wherein the heating wire (22a, 22b) is configured in the form of a plastic-coated heating coil (21) around an inner lumen of the tube component (11).

3. The assembly according to claim 1, wherein the connection piece (22) extends radially from a longitudinal axis (L) of the connector (37).

4. The assembly according to claim 1, wherein the protective housing (28) is configured such that a plurality of heating wire end portions (22a, 22b) are electrically contacted therein to associated supply line end portions (26a, 26b).

5. The assembly according to claim 4, wherein the protective housing (28) has at least one separating web (31) for insulation between the heating wire end portions (22a, 22b) or the supply line end portions (26a, 26b).

6. The assembly according to claim 1, wherein the protective housing (28) has a cover (32) which is connected to a base body (30) of the protective housing (28).

7. The assembly according to claim 6, comprising a solid-body hinge (33) between the cover (32) and the base body (30).

8. The assembly according to claim 1, comprising an overmolding body (39) which is arranged such as to support a tube wall (20) of the end portion from the inside when the connector (38) is molded to the end portion of the tube component (11).

9. The assembly according to claim 8, wherein the overmolding body (39) has a radially projecting collar (40) for axial fixation of the overmolding body (39) in the connector (38) during overmolding.
